(19) United States
(12) Patent Application Publication
(10) Pub. No.: US 2024/0100477 A1
(43) Pub. Date: Mar. 28, 2024

(54) METHODS AND SYSTEMS FOR PROCESSING GAS STREAMS

(71) Applicant: SPV APEX 1, Inc., Menlo Park, CA (US)

(72) Inventors: Joshua A. Silverman, Los Altos Hills, CA (US); Erik Scher, San Francisco, CA (US); Robert B. Jackson, Stanford, CA (US); Guido Radaelli, Pleasant Hill, CA (US)

(21) Appl. No.: 18/451,484

(22) Filed: Aug. 17, 2023

Related U.S. Application Data
(63) Continuation of application No. PCT/US2022/016880, filed on Feb. 17, 2022.
(60) Provisional application No. 63/151,017, filed on Feb. 18, 2021.
(60) Provisional application No. 63/151,016, filed on Feb. 18, 2021.
(60) Provisional application No. 63/151,021, filed on Feb. 18, 2021.

(51) Int. Cl. B01D 53/85; B01D 53/72; C12N 1/20

(52) U.S. Cl. CPC B01D 53/85; B01D 53/72; C12N 1/20; B01D 2251/95; B01D 2255/804; B01D 2257/7025

(57) ABSTRACT

Described herein are methods and systems for processing gas streams. The gas streams may comprise a methane-containing gas stream, such as an exhaust stream. The systems and methods of the present disclosure may process the methane-containing gas stream using one or more processing units including a biological filtration unit and a thermal oxidizer to generate an output stream which has a lower concentration of methane than the methane-containing gas stream.

### CROSS-REFERENCE

This application is a continuation application of International Patent Application No. PCT/US2022/016880, filed Feb. 17, 2022, which claims the benefit of U.S. Provisional Application No. 63/151,017, filed Feb. 18, 2021, U.S. Provisional Application No. 63/151,016, filed Feb. 18, 2021, and U.S. Provisional Application No. 63/151,021, filed Feb. 18, 2021 which applications are incorporated herein by reference.

### BACKGROUND

Methane emissions represent a significant portion of global greenhouse gas emissions. One ton of methane is equivalent to approximately up to 86 tons of carbon dioxide with respect to its global warming potential over a 20-year period. Moreover, methane has about a 12-year half-life in the atmosphere and is increasing worldwide at an annual rate of around one half of one percent (0.5%) per year. Methane emissions originate from both anthropogenic and non-anthropogenic sources, and many natural emissions sources such as wetlands and permafrost are exacerbated by overall global warming.

### SUMMARY

Various technologies and measures have been deployed in an attempt to control and reduce methane emissions. However, methane emissions are currently at their highest levels ever recorded, and rapidly increasing. In particular, most methane emissions to the atmosphere originate in the form of gas streams which are too dilute for efficient mitigation by current technologies. Thus, recognized herein is a need for new technologies and methods to encourage and facilitate the reduction of methane emissions with a particular focus on dilute methane streams.

The present disclosure provides methods and systems for processing a gas stream, such as a methane-containing gas stream. The systems of the present disclosure may be configured to receive and process an input stream and reduce a concentration or amount of methane from the input stream to generate an output stream which has a reduced methane content than the input stream. The present disclosure provides methods and systems for promoting a decrease in methane emissions. The system of the present disclosure may also be configured to measure methane concentrations, generate data blocks indicating a quantity of lowered methane content, and facilitate the transactions of carbon credits associated with methane reducing activities on a blockchain database.

An aspect of the present disclosure provides a system for processing a methane-containing gas stream, comprising: (a) a biological filtration unit configured to (i) receive the methane-containing gas stream and (ii) process methane from the methane-containing gas stream to generate a first stream; and (b) a thermal oxidizer in fluid communication with the biological filtration unit, wherein the thermal oxidizer is configured to process at least a portion of methane in the first stream to yield a second stream which has a lower concentration of methane than the methane-containing gas stream.

In some embodiments, the system further comprises one or more additional units between the biological filtration unit and the thermal oxidizer. In some embodiments, the thermal oxidizer is fluidically connected in series with the biological filtration unit. In some embodiments, the thermal oxidizer is a regenerative thermal oxidizer. In some embodiments, the biological filtration unit is operated at a temperature between about 4° C. and about 60° C. In some embodiments, the biological filtration unit is operated under a pressure between about 0.75 atmospheric pressure (atm) and about 1.25 atm. In some embodiments, thermal oxidizer is operated at a temperature between about 600° C. and about 1200° C. In some embodiments, the biological filtration unit comprises biological materials. In some embodiments, the biological materials comprise microorganisms. In some embodiments, the microorganisms comprise Methylococcus capsulatus, Methylosinus trichosporium, or any combination or variant thereof. In some embodiments, the biological filtration unit permits the biological materials to react with methane in the methane-containing gas stream under conditions such that at least a portion of the methane is oxidized. In some embodiments, the biological filtration unit produces methylotrophic bacterial biomass. In some embodiments, the system further comprises a gas monitoring unit upstream of the biological filtration unit, wherein the gas monitoring unit is configured to measure a composition of the methane-containing gas stream. In some embodiments, the composition comprises a concentration of carbon dioxide, methane, or a combination thereof in the methane-containing gas stream. In some embodiments, the system further comprises an additional gas monitoring unit configured to measure a composition of the first stream. In some embodiments, the system further comprises an additional gas monitoring unit configured to measure a composition of the second stream. In some embodiments, the system further comprises at least one gas mixing unit. In some embodiments, the at least one gas mixing unit is upstream of the biological filtration unit or the thermal oxidizer. In some embodiments, the at least one gas mixing unit comprises a first gas mixing unit upstream of the biological filtration unit, and a second gas mixing unit upstream of the thermal oxidizer. In some embodiments, the at least one gas mixing unit is configured to mix air or an accelerant with the methane-containing gas stream, or the first stream, to maintain a methane concentration in the methane-containing gas stream or the first stream. In some embodiments, the at least one gas mixing unit is configured to mix air or an accelerant with the methane containing gas stream, or the first stream, to maintain a heating potential of the methane-containing gas stream or the first stream. In some embodiments, the accelerant comprises methane, propane or any combination or variant thereof. In some embodiments, the at least one gas mixing unit is in fluid communication with the gas monitoring unit. In some embodiments, the at least one gas mixing unit is configured to mix the air or the accelerant with the methane-containing gas stream or the first stream in response to a measurement received from the gas monitoring unit. In some embodiments, the system further comprises a gas switch configured to direct gas transfer within the system. In some embodiments, the system further comprises a condenser in fluid communication with the biological filtration unit or the thermal oxidizer. In some embodiments, the condenser is downstream of the biological filtration unit. In some embodiments, the condenser is upstream of the thermal oxidizer. In some embodiments, the condenser is configured to condense at least a portion of the first stream or the second stream to generate a water stream. In some embodiments, the system further comprises a humidifier in fluid communication with the biological filtration unit. In some embodiments, the humidifier is upstream of the biological filtration unit. In some embodiments, the humidifier is configured to maintain sufficient moisture for the survival of the microorganisms in the biological filtration unit. In some embodiments, the system further comprises a controller and data logging unit configured to direct and control an operation of the system. In some embodiments, the system further comprises a geo-positioning subsystem.

An aspect of the present disclosure provides a method for processing a methane-containing gas stream, comprising: (a) directing the methane-containing gas stream to a biological filtration unit that processes methane from the methane-containing gas stream to generate a first stream; and (b) using a thermal oxidizer to process at least a portion of methane in the first stream to yield a second stream which has a lower concentration of methane than the methane-containing gas stream.

In some embodiments, the methane-containing gas stream comprises less than or equal to about 1 vol % methane. In some embodiments, the first stream comprises less than or equal to about 1 vol % methane. In some embodiments, the second stream comprises less than or equal to about 500 parts per million (ppm) methane. In some embodiments, the second stream comprises less than or equal to about 100 ppm methane. In some embodiments, the second stream comprises less than or equal to about 50 ppm methane. In some embodiments, the second stream comprises less than or equal to about 5 ppm methane. In some embodiments, the second stream comprises less than or equal to about 1 ppm methane.

An aspect of the present disclosure provides a non-transitory computer readable medium comprising machine executable code that, upon execution by one or more computer processors, implements any of the methods above or elsewhere herein.

An aspect of the present disclosure provides a system comprising one or more computer processors and computer memory coupled thereto. The computer memory comprises machine executable code that, upon execution by the one or more computer processors, implements any of the methods above or elsewhere herein.

An aspect of the present disclosure provides a system for processing a methane-containing gas stream, comprising a processing unit, comprising: (a) a matrix for binding at least a portion of methane from the methane-containing gas stream to yield bound methane; and (b) a catalyst configured to subject at least a portion of the bound methane to a reaction that decreases an amount or concentration of the bound methane.

In some embodiments, the processing unit further comprises a heating element. In some embodiments, the heating element is configured to heat the matrix to a temperature such that at least a portion of the bound methane is oxidized, thereby decreasing the amount or concentration of the bound methane. In some embodiments, the processing unit is configured to (a) receive the methane-containing gas stream; (b) bind the at least the portion of the methane from the methane-containing gas stream to yield the bound methane; and (c) subject the at least the portion of bound methane to the reaction with aid of the catalyst to produce an output stream which has a lower concentration of methane than the methane-containing gas stream. In some embodiments, the system further comprises a gas monitoring unit configured to measure a composition of the methane-containing gas stream. In some embodiments, the system further comprises an additional gas monitoring unit configured to measure a composition of the output stream. In some embodiments, the system further comprises a temperature control unit configured to adjust a temperature of the processing unit. In some embodiments, the temperature control unit is configured to increase a temperature above a predetermined value to initiate the reaction upon an additional gas stream being directed into the processing unit. In some embodiments, the additional gas stream comprises air, an accelerant, or any combination or variant thereof. In some embodiments, the methane-containing gas stream is an exhaust stream. In some embodiments, the methane-containing gas stream comprises less than or equal to about 5 vol % methane. In some embodiments, the methane-containing gas stream comprises greater than or equal to about 1 ppm methane. In some embodiments, the system further comprises an additional processing unit connected in parallel with the processing unit. In some embodiments, the additional processing unit comprises an additional matrix and an additional catalyst. In some embodiments, the matrix and the additional matrix are the same. In some embodiments, the matrix and the additional matrix are different. In some embodiments, the catalyst and the additional catalyst are the same. In some embodiments, the catalyst and the additional catalyst are different. In some embodiments, the matrix comprises a zeolite, alumina, cordierite, silica, a metal foil, or a metal organic framework. In some embodiments, the catalyst comprises Pd, Pt, Ni, Cu, Co, or any combination thereof. In some embodiments, the system further comprises a switching unit configured to alternate gas flow between the methane-containing gas stream and an additional gas stream to the processing unit or the additional processing unit. In some embodiments, the additional gas stream comprises air. In some embodiments, the additional gas stream further comprises an accelerant. In some embodiments, the accelerant comprises methane or propane.

An aspect of the present disclosure provides a method for processing a methane-containing gas stream, comprising: (a) directing the methane-containing gas stream into a processing unit comprising a matrix and a catalyst; (b) using the matrix to bind at least a portion of methane from the methane-containing gas stream to yield bound methane; and (c) using the catalyst to subject at least a portion of the bound methane to a reaction that decreases an amount or concentration of the bound methane.

In some embodiments, the reaction is a combustion reaction of methane. In some embodiments, (c) comprises oxidizing the at least the portion of the bound methane in the reaction, thereby regenerating the processing unit. In some embodiments, in (b), greater than or equal to about 50% of methane in the methane-containing gas stream is bound to the matrix. In some embodiments, in (c), greater than or equal to about 50% of the bound methane is reacted in the reaction. In some embodiments, (c) comprises directing an additional gas stream to the processing unit to react with the at least the portion of the bound methane. In some embodiments, the method further comprises directing the methane-containing gas stream into an additional processing unit connected to the processing unit in parallel. In some embodiments, the additional processing unit comprises one or more of an additional matrix, an additional catalyst, and an additional heating element. In some embodiments, the method further comprises alternating gas flow between the methane-containing gas stream and the additional gas stream to the processing unit and the additional processing unit. In some embodiments, the method further comprises (d) using the additional matrix to bind additional methane from the methane-containing gas stream to yield additional bound methane. In some embodiments, in (d), greater than or equal to about 50% of additional methane in the methane-containing gas stream is bound to the additional matrix. In some embodiments, the method further comprises (e) subjecting the additional bound methane to an additional reaction with aid of the additional catalyst to oxidize at least a portion of the additional bound methane. In some embodiments, in (e), greater than or equal to about 50% of the additional bound methane is oxidized in the additional reaction. In some embodiments, the method further comprises repeating (b)-(e) one or more times to yield an output stream which has a lower concentration of methane than the methane-containing gas stream.

An aspect of the present disclosure provides a computer-implemented method for promoting a decrease in methane emissions, comprising: (a) receiving, by one or more computer processors in operative communication with a quantified destruction system (QDS), methane content data from said QDS, wherein said QDS (i) lowers a methane content of a gas stream to yield a lowered methane content, (ii) measures said methane content data comprising said methane content of said gas stream, and (iii) transmits said methane content data to said one or more computer processors; (b) encrypting, by said one or more computer processors, a data block comprising lowered methane content data derived from said methane content data and a QDS identifier, to generate encrypted data; and (c) storing said encrypted data on a blockchain database.

In some embodiments, the QDS is a quantified destruction device (QDD). In some embodiments, the gas stream is an input gas stream received by said QDS. In some embodiments, the methane content data comprises said methane content of said input gas stream and a methane content of an output gas stream generated by said QDS. In some embodiments, the QDS further lowers an amount or concentration of one or more additional components in said input gas stream. In some embodiments, the one or more additional components comprises carbon dioxide, nitrogen oxides, other volatile organic compounds, or any combination thereof. In some embodiments, the QDS further tracks said amount or concentration of said one or more additional components. In some embodiments, the QDS further measures gas content data comprising said amount or concentration of said one or more additional components and transmits said gas content data to said one or more computer processors. In some embodiments, the said lowered methane content data comprises data on emission reductions of said methane content between said input gas stream and said output gas stream. In some embodiments, the method further comprises tracking and recording said emission reductions from said QDS. In some embodiments, the method further comprises tracking an exchange of emission units based on said lowered methane content data between users of said blockchain database. In some embodiments, the method further comprises, processing, by said one or more computer processors, said lowered methane content data to determine a carbon credit. In some embodiments, the method further comprises issuing said carbon credit as digital tokens of said blockchain database to a user associated with said QDS. In some embodiments, the encrypted data stored on said blockchain database comprises data on said issuing of said digital tokens. In some embodiments, the method further comprises tracking a transaction of said carbon credit between users of said blockchain database. In some embodiments, the transaction is digitally signed by a trusted third-party. In some embodiments, the method further comprises disseminating a digital wallet to said QDS. In some embodiments, the method further comprises storing said digital wallet in said blockchain database. In some embodiments, the method further comprises associating said carbon credit with said digital wallet and storing said carbon credit in said digital wallet. In some embodiments, the method further comprises recording and validating new entries on said blockchain database. In some embodiments, the data block further comprises data on geo-positioning information of said QDS, and a time stamp. In some embodiments, the QDS identifier is unique to said QDS amongst a plurality of QDSs. In some embodiments, the blockchain database comprises a network of distributed nodes in operative communication with said one or more computer processors.

An aspect of the present disclosure provides a system for promoting a decrease in methane emissions, comprising: (a) a quantitative destruction system (QDS) configured to (i) lower a methane content of a gas stream to yield a lowered methane content, (ii) measure methane content data comprising said methane content of said gas stream, and (iii) transmit said methane content data, wherein said QDS is associated with a QDS identifier unique to said QDS; and (b) one or more computer processors in operative communication with said QDS, wherein said one or more computer processors are individually or collectively configured to: (i) receive said methane content data, (ii) encrypt a data block comprising said QDS identifier and lowered methane content data derived from said methane content data, to generate encrypted data, and (iii) store said encrypted data on a blockchain database.

In some embodiments, the QDS is a part of a plurality of QDSs in fluid communication with a plurality of input gas streams, wherein said one or more computer processors are in operative communication with said plurality of QDSs, and wherein each QDS is associated with a respective QDS identifier unique to said each QDS amongst said plurality of QDSs. In some embodiments, the blockchain database comprises a platform for recording and validating new entries on said blockchain database. In some embodiments, the one or more computer processors are further configured to process said lowered methane content data to determine a carbon credit. In some embodiments, the one or more computer processors are further configured to issue said carbon credit as digital tokens of said blockchain database to a user associated with said QDS. In some embodiments, the encrypted data stored on said blockchain database comprises data on issuance of said digital tokens. In some embodiments, the blockchain database comprises an additional platform tracking a transaction of said carbon credit between users of said blockchain database. In some embodiments, the transaction is digitally signed by a trusted third-party. In some embodiments, the blockchain database comprises a platform for tracking an exchange of emission units, based on said lowered methane content data, between users of said blockchain database. In some embodiments, the system further comprises a blockchain verification module configured to generate one or more binary strings to identify a transaction on said blockchain database. In some embodiments, a first binary string of said one or more binary strings identifies a current blockchain node as a transaction recipient and wherein a second binary string of said one or more binary strings identifies said current blockchain node as a transaction sender. In some embodiments, the QDS comprises one or more of a methane input measurement module, a methane destruction module, a methane output measurement module, a carbon dioxide (CO₂) output measurement module, a geo-positioning module, and a time recording module. In some embodiments, the encrypted data on said blockchain database is accessible by a trusted third-party. In some embodiments, the or more computer processors are further configured to retire or eliminate a digital token according to predetermined criteria. In some embodiments, the predetermined criteria comprise a threshold for the elapsed time from the date said methane content is reduced by said QDS. In some embodiments, the predetermined criteria comprise a voluntary decision by an owner of a digital token.

An aspect of the present disclosure provides a non-transitory computer readable medium comprising machine executable code that, upon execution by one or more computer processors, implements any of the methods above or elsewhere herein.

An aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example system comprising a biological filtration unit and a thermal oxidizer for processing a methane-containing gas stream;

FIG. 2 shows a computer system that is programmed or otherwise configured to implement methods provided herein; and

FIG. 3 shows an example system comprising a plurality of processing units in parallel for processing a methane-containing gas stream.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “no more than,” “less than,” or “less than or equal to” precedes the first numerical value in a series of two or more numerical values, the term “no more than” or “less than” applies to each one of the numerical values in that series of numerical values.

The term “about” or “nearly” as used herein generally refers to within (plus or minus) 15%, 10%, 9%, 8%, 7%, 6%, 5%, 4%, 3%, 2%, or 1% of a designated value.

As used herein, the singular forms “a”, “an”, and “the” include plural references unless the context clearly dictates otherwise.

In some aspects, provided herein is a system for processing a gas stream, such as a methane-containing gas stream. The system may comprise one or more units in fluid communication or operably coupled with one another. For example, the system may comprise a biological filtration unit and a thermal oxidizer. The thermal oxidizer may be in in fluid communication with the biological filtration unit. The biological filtration unit and the thermal oxidizer may be connected in series. The biological filtration unit may be upstream, or downstream, of the thermal oxidizer. The biological filtration unit may be configured to receive an input stream, such as a methane-containing gas stream, and process at least a portion of methane from the methane-containing gas stream to generate a first stream comprising methane. The methane-containing gas stream may be an exhaust stream of another system. The thermal oxidizer may be configured to receive at least a portion of the first stream from the biological filtration unit and process at least a portion of methane in the first stream to yield a second stream. The second stream may have a lower concentration of methane than the methane-containing stream.

The system may comprise one or more additional units (e.g., greater than or equal to about 1, 2, 3, 4, 5, 6, 7, 8, 9, 10 additional units or more). The one or more additional units may comprise a condenser, a humidifier, a gas monitoring unit, a gas switch, a gas mixing unit, a temperature control unit, a heating unit, and a geo-positioning unit. The additional units may be between the biological filtration unit and/or the thermal oxidizer. The biological filtration unit, the thermal oxidizer and the additional units may be connected with one another in series. In some cases, some of the additional units are upstream or downstream of one or both of the biological filtration unit and the thermal oxidizer. The thermal oxidizer may be a regenerative thermal oxidizer that captures heat from the exhaust stream of another system. The biological filtration unit may be operated at a temperature which is greater than or equal to about 4° C., 6° C., 8° C., 10° C., 15° C., 20° C., 25° C., 30° C., 35° C., 40° C., 45° C., 50° C., 55° C., 60° C., 65° C., 70° C., or higher. The biological filtration unit may be operated at a temperature which is less than or equal to about 80° C., 75° C., 70° C., 65° C., 60° C., 55° C., 50° C., 45° C., 40° C., 35° C., 30° C., 25° C., 20° C., 15° C., 10° C., 7° C., 5° C., 3° C., or lower. The biological filtration unit may be operated at a temperature between any two of the values described above, e.g., between about 4° C. and about 60° C.

The biological filtration unit may be operated at a pressure which is greater than or equal to about 0.20 atmospheric pressure (atm), 0.25 atm, 0.30 atm, 0.35 atm, 0.40 atm, 0.45 atm, 0.50 atm, 0.55 atm, 0.60 atm, 0.65 atm, 0.70 atm, 0.75 atm, 0.80 atm, 0.85 atm, 0.9 atm, 0.95 atm, 1.00 atm, 1.05 atm, 1.10 atm, 1.15 atm, 1.20 atm, 1.25 atm, 1.50 atm, or greater. The biological filtration unit may be operated at a pressure which is lower than or equal to about 10 atm, 5 atm, 3 atm, 2 atm, 1.75 atm, 1.50 atm, 1.45 atm, 1.40 atm, 1.35 atm, 1.30 atm, 1.25 atm, 1.20 atm, 1.15 atm, 1.10 atm, 1.05 atm, 1.00 atm, 0.95 atm, 0.90 atm, 0.85 atm, 0.80 atm, 0.75 atm, 0.70 atm, 0.65 atm, 0.60 atm, 0.55 atm, 0.50 atm, or lower. The biological filtration unit may be operated between any two of the pressures described above, e.g., between about 0.75 atm and about 1.25 atm.

The thermal oxidizer may be operated at a temperature which is greater than or equal to about 200° C., 250° C., 300° C., 350° C., 400° C., 450° C., 500° C., 550° C., 600° C., 650° C., 700° C., 750° C., 800° C., 850° C., 900° C., 950° C., 1000° C., 1050° C., 1100° C., 1150° C., 1200° C., 1250° C., 1300° C., or greater. The thermal oxidizer may be operated at a temperature which is lower than or equal to about 1500° C., 1400° C., 1300° C., 1250° C., 1200° C., 1150° C., 1100° C., 1050° C., 1000° C., 950° C., 900° C., 850° C., 800° C., 750° C., 700° C., 650° C., 600° C., 500° C., 400° C., or lower. The thermal oxidizer may be operated between any two of the temperatures described above, e.g., between about 600° C. and about 1200°.

The biological filtration unit may comprise biological materials. The biological materials may comprise bioactive materials. The biological materials may comprise microorganisms (e.g., bacteria), or a consortium thereof. The biological materials may metabolize methane as a primary or sole source of carbon and energy, thereby reducing an amount or a concentration of methane as originally included in a gas stream.

The microorganisms may comprise methanotrophs, non-methanotrophs, or a combination thereof. Non-limiting examples of methanotrophs may comprise Methylococcus capsulatus (Bath), Methylomonas sp. 16a, Methylosinus trichosporium OB3b, Methylosinus sporium, Methylocystis parvus, Methylomonas methanica, Methylomonas albus, Methylobacter capsulatus, Methylobacterium organophilum, Methylomonas sp. AJ3670, Methylomicrobium alcaliphilum, Methylocella silvestris, Methylacidiphilum infernorum, Methylibium petroleiphilum, or any combination or variant thereof. In some embodiments, non-methanotroph microorganisms may be engineered to express a functional methane monooxygenase enzyme enabling the oxidation of methane with or without further metabolism of the methane. In other embodiments, the biological filtration unit may comprise a microbiological consortium comprising both methanotroph and non-methanotroph microorganisms whereby the non-methanotroph microorganisms promote the growth and health of the methanotroph culture.

The biological filtration unit may permit the biological materials to react with methane in the methane-containing gas stream. At least a portion of methane may be oxidized in this reaction with the biological materials. As a result of this reaction, the biological filtration unit may produce biomass. The biomass may be subject to one or more subsequent processing steps.

The system may comprise one or more gas monitoring units. A gas monitoring unit may be upstream or downstream of the biological filtration unit and/or thermal oxidizer. The gas monitoring unit may be configured to measure a gas composition, such as a concentration of carbon dioxide, methane, or a combination thereof. The gas monitoring unit may be configured to measure a composition of the first stream, the second stream, or the methane-containing gas stream. For example, a first gas monitoring unit may be upstream of the biological filtration unit, a second gas monitoring unit may be downstream of the biological filtration unit and upstream of the thermal oxidizer, and a third gas monitoring unit may be downstream of the biological filtration unit and thermal oxidizer. The gas monitoring unit may be configured to continuously monitor the gas composition in real-time. The gas monitoring unit may be configured to continuously transmit the measured gas composition in real time, to one or more computer processors.

The system may comprise one or more gas mixing units. One or more of the gas mixing units may be in fluid connection and operably coupled to one or more of the gas monitoring units. For example, the system may comprise a first gas mixing unit upstream of the biological filtration unit, and a second gas mixing unit upstream of the thermal oxidizer. One or more of the gas mixing units may mix air or an accelerant with the methane-containing gas stream or the first stream, to maintain a methane concentration or heating potential in the methane-containing gas stream or first stream, based on the concentration measured by of one or more of the gas monitoring units. The accelerant may comprise methane, ethane, propane, butane, methanol, ethanol, propanol, butanol, formate, formaldehyde, acetate, acetaldehyde or any combination or variant thereof.

The system may comprise a gas switch configured to direct gas transfer within the system. The gas switch may alternate the flow of methane-containing gas stream to the biological filtration unit, the thermal oxidizer, or the one or more additional units. The gas switch may be configured to bypass the biological filtration units, the thermal oxidizer, or the one or more additional units. For example, the gas switch may switch the methane-containing gas stream to bypass the biological filtration unit and flow directly to the thermal oxidizer unit. The gas switch may be configured to switch the flow of methane-containing gas stream to bypass the biological filtration unit at the direction of a signal from another unit, e.g. a gas monitoring unit. The gas switch may also be configured to switch the flow of methane-containing gas stream to bypass the biological filtration unit, and switch back, on a pre-set or adjustable timing schedule. The gas switch may also be configured to be operated manually.

The system may comprise a condenser. The condenser may be in fluid communication with the biological filtration unit, the thermal oxidizer, and/or the one or more additional units. The condenser may be configured to condense at least a portion of the first stream or second stream to generate a water stream. The condenser may be upstream or downstream of the biological filtration unit, thermal oxidizer, and/or the one or more additional units. For example, the condenser may be downstream of the biological filtration unit and upstream of the thermal oxidizer.

The system may comprise a humidifier. The humidifier may be in fluid communication with the biological filtration unit, the thermal oxidizer, and/or the one or more additional units. For example, the humidifier may be in fluid communication with the biological filtration unit, thermal oxidizer, and condenser. The humidifier may receive, as an input, a water stream produced by the condenser unit. The humidifier may be upstream or downstream of the biological filtration unit, thermal oxidizer, and/or the one or more additional units. For example, the humidifier may be upstream of the biological filtration unit and downstream of the condenser. The humidifier may be configured to maintain a level of moisture sufficient for the survival of the microorganisms within the biological filtration unit.

The system may comprise one or more sensors. The one or more sensors may comprise a humidity sensor. The humidifier may be in fluid communication with a humidity sensor within the biological filtration unit. The humidifier may be configured to maintain a humidity level which is greater than or equal to about 3 weight % (wt %), 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 30 wt %, or more. In some cases, weight % is the weight of water per unit of air. The humidifier may be configured to maintain a humidity level which is less than or equal to about 40 wt %, 35 wt %, 30 wt %, 25 wt %, 24 wt %, 23 wt %, 22 wt %, 21 wt %, 20 wt %, 19 wt %, 18 wt %, 17 wt %, 16 wt %, 15 wt %, 14 wt %, 13 wt %, 12 wt %, 11 wt %, 10 wt %, 5 wt %, or less. The humidifier may be configured to maintain a level of moisture between any two of the moisture levels above, e.g. between 15 wt % and 20 wt %,

The system may comprise a controller and data logging unit. The controller and data logging unit may be configured to direct and control an operation of the system. For example, the controller may be configured to direct and/or control the gas switch, gas mixing unit, heating element, biological filtration unit, thermal oxidizer, humidifier, condenser, or any one of the one or more additional units. The data logging unit, for example, may log the measured values from the gas monitoring unit, the temperature control unit, any of the one or more sensors, or any of the one or more additional units. The controller or data logging unit may transmit to one or more computer processors in real time. The computer processors may then transmit one or more signals to the one or more additional units, (e.g. the gas mixing unit, temperature control unit, one or more of the controllers, or one of the one or more additional units), to effect a change to the operation of the system.

The system may comprise a geo-positioning sub-system. The geo-positioning sub-system may be operably coupled to the biological filtration unit, thermal oxidizer, and/or one or more additional units. The geo-positioning sub-system may be configured to determine a location of the sub-system. The geo-positioning sub-system may be configured to generate a time stamp for that determined location and transmit the paired time stamp and location to one or more computer processors or data logging units. For example, the geo-positioning sub-system may determine the sub-system is located at location A, during the date or time range X, and the sub-system can transmit the paired data of location A, during time X, to one or more computer processors. The geo-positioning sub-system may be configured to generate an alert, by way of transmitting a signal to one or more computer processors, upon a change in the location of the geo-positioning sub-system. For example, the geo-positioning sub-system may generate a location A, and when the sub-system is relocated to location B, the sub-system can alert one or more computer processors to that change in location.

In some aspects, provided herein is a method for processing a gas stream, such as a methane-containing gas stream. The method may comprise directing the methane-containing gas stream to a biological filtration unit. The biological filtration unit can process methane from the methane-containing gas stream to generate a first stream. The method may comprise using a thermal oxidizer to process a portion of methane in the first stream. The thermal oxidizer can yield a second stream which has a lower concentration of methane than the methane-containing stream. The methane-containing stream may comprise less than or equal to about 1 vol % methane. The output stream may comprise a concentration of methane that is less than or equal to about 2000 parts per million (ppm), 1500 ppm, 1000 ppm, 750 ppm, 500 ppm, 400 ppm, 300 ppm, 200 μm, 150 ppm, 100 ppm, 75 ppm, 50 ppm, 25 ppm, 20 ppm, 15 ppm, 10 ppm, 7 ppm, 4 ppm, 1 ppm, or lower. The output stream may comprise a concentration of methane that is greater than or equal to about 0.1 ppm, 0.5 ppm, 1 ppm, 3 ppm, 6 ppm, 10 ppm, 20 ppm, 50 ppm, 100 ppm, 500 ppm, 1000 ppm, or greater. The output stream may comprise a concentration of methane that is between any two of the concentrations described above, e.g. between 1 ppm and 500 ppm.

The first stream may comprise less than or equal to about 1 vol % methane. The second stream may comprise less than or equal to about 500 ppm, 100 ppm, 50 ppm, 5 ppm, 1 ppm, or lower methane.

In some aspects, provided herein is a system for processing a methane-containing gas stream. The system may comprise a processing unit, comprising a matrix and a catalyst. The matrix may be configured to bind a portion of methane from the methane-containing gas stream. The catalyst may be configured to subject a portion of bound methane to a reaction (e.g. combustion or oxidation reaction) that decreases an amount or concentration of the bound methane in the matrix. The portion of methane in the methane-containing gas stream that is bound by the matrix in the processing unit, may be greater than or equal to about 1 vol %, 3 vol %, 5 vol %, 10 vol %, 15 vol %, 20 vol %, 25 vol %, 30 vol %, 35 vol %, 40 vol %, 45 vol %, 50 vol %, 55 vol %, 60 vol %, 65 vol %, 70 vol %, 75 vol %, 80 vol %, 85 vol %, 90 vol %, 95 vol %, or greater. The portion of methane in the methane-containing gas stream that is bound by the matrix in the processing unit may be less than or equal to about 100 vol %, 98 vol %, 95 vol %, 90 vol %, 85 vol %, 80 vol %, 75 vol %, 70 vol %, 65 vol %, 60 vol %, 55 vol %, 50 vol %, 45 vol %, 40 vol %, 35 vol %, 30 vol %, 25 vol %, 20 vol %, 15 vol %, 10 vol %, 5 vol %, or lower. The portion of methane in the methane-containing gas stream that is bound by the matrix in the processing unit may be between any two of the portions above, e.g. between 30 vol % and 65 vol %.

The processing unit may comprise a heating element. The heating element may be configured to heat the matrix to a temperature such that at least a portion of the bound methane is oxidized. The system, or processing unit, may comprise a temperature control unit configured to adjust a temperature of the processing unit. The temperature control unit may be operably coupled to the heating element within the processing unit. The temperature control unit can be configured to increase a temperature of the processing unit above a predetermined value to initiate the reaction of methane within the processing unit. The processing unit, or matrix within the processing unit, may be heated to a temperature which is greater than or equal to about 100° C., 150° C., 200° C., 250° C., 300° C., 350° C., 400° C., 450° C., 500° C., 550° C., 600° C., 650° C., 700° C., 750° C., 800° C., 850° C., 900° C., 950° C., 1000° C., 1050° C., 1100° C., 1150° C., 1200° C., 1250° C., 1300° C., or greater. The processing unit, or matrix within the processing unit, may be heated to a temperature which is lower than or equal to about 1500° C., 1400° C., 1300° C., 1250° C., 1200° C., 1150° C., 1100° C., 1050° C., 1000° C., 950° C., 900° C., 850° C., 800° C., 750° C., 700° C., 650° C., 600° C., 500° C., 400° C., 300° C., 200° C., 100° C., or lower. The processing unit, or matrix within the processing unit, may be heated to a temperature which is between any two of the temperatures described above, e.g. between about 600° C. and about 1200°.

The processing unit can receive an additional gas stream. The temperature control unit can be configured to initiate the temperature increase upon a specified process step. For example, the temperature control unit may initiate a temperature increase simultaneous with the additional gas stream being directed into the processing unit. The additional gas stream may comprise air, an accelerant, or any combination thereof. The accelerant may comprise methane, ethane, propane, butane or any combination or variant thereof.

The system may comprise a processing unit configured to (i) receive the methane-containing gas stream, (ii) bind at least a portion of methane from the methane-containing gas stream to yield bound methane, and (iii) subject at least a portion of the bound methane to a reaction with aid of the catalyst. The catalyst may comprise Pd, Pt, Rh, S, Ni, Cu, Co, another compound, or any combination thereof. This processing unit can produce an output stream which has a lower concentration of methane than the methane-containing gas stream. The methane-containing gas stream may be an exhaust stream of another system. The methane-containing gas stream may comprise less than or equal to about 20 vol %, 15 vol %, 10 vol %, 5 vol %, 4 vol %, 3 vol %, 2 vol %, 1 vol %, 0.5 vol %, 0.1 vol %, or lower methane. The methane-containing gas stream may comprise greater than or equal to about 0.1 ppm, 0.5 ppm, 1 ppm, 2 ppm, 3 ppm, 4 ppm, 5 ppm, 10 ppm, 15 ppm, 20 ppm, 50 ppm, 100 ppm, 500 ppm, 1000 ppm, or greater methane.

The output stream may comprise a concentration of methane that is less than or equal to about 2000 ppm, 1500 ppm, 1000 ppm, 750 ppm, 500 ppm, 400 ppm, 300 ppm, 200 ppm, 150 ppm, 100 ppm, 75 ppm, 50 ppm, 25 ppm, 20 ppm, 15 ppm, 10 ppm, 7 ppm, 4 ppm, 1 ppm, or lower. The output stream may comprise a concentration of methane that is greater than or equal to about 0.1 ppm, 0.5 ppm, 1 ppm, 3 ppm, 6 ppm, 10 ppm, 20 ppm, 50 ppm, 100 ppm, 500 ppm, 1000 ppm, or greater. The output stream may comprise a concentration of methane that is between any two of the concentrations described above, e.g. between 1 ppm and 500 ppm.

The system may comprise the processing unit and an additional processing unit. The processing unit and the additional processing unit may be connected in parallel. The additional processing unit may comprise an additional matrix, an additional catalyst, and an additional heating unit. The additional heating unit may be the same as or different from the heating unit in the original processing unit. The original matrix and the additional matrix may be the same or different. The original catalyst and the additional catalyst may be the same or different.

Non-limiting examples of catalysts may comprise Pd, Pt, Rh, S, Ni, Cu, Co, another compound, or any combination thereof. The catalysts may comprise Rh or Au metal oxide catalysts including single metal oxides, (e.g. CuO, MgO, Co₃O₄), or mixed metal oxides such as perovskites, hexaaluminate, or doped metal oxides. The perovskites may be nanostructured perovskite oxides. The catalysts may comprise noble metal catalysts, e.g. Pd or Pt-based catalysts. The noble metal catalysts may be supported with silica or alumina. The catalysts may comprise a bi-metallic system combining Pd and Pt-based catalysts.

Any suitable matrices may be used. The matrices may comprise a membrane or a framework. The matrices may comprise a mixed membrane material. Any of the matrices may comprise a zeolite, alumina, cordierite, silica, a metal foil, or a metal organic framework. The matrixes may comprise a zeolitic imidazolate framework (ZIF) material. The ZIF material may comprise a network of homogeneous transition metal or heterogeneous transition metals linked by a homogeneous or heterogeneous linking moiety. Non-limiting examples of transition metals may comprise Sc, Ti, V, CR, MN, FE, CO, Ni, Cu, Zn, Y, Zr, Nb, Mo, Tc, Ru, Rh, Pd, Ag, Cd, Lu, Hf, Ta, W, Re, Os, Ir, Pt, Au, Hg, Lr, Rf, db, Sg, Bh, Hw, Mt, Ds, Rg, or Uub. The linking moieties may comprise organic linkers comprising nitrogen, sulfur or oxygen organic molecules (e.g. imidazolate units). The mixed membrane material may comprise a ZIF-containing layer. The ZIF containing layer may comprise greater than or equal to about 1 vol %, 5 vol %, 10 vol % 15 vol %, 20 vol %, 25 vol %, 30 vol %, 35 vol %, 40 vol %, 45 vol %, or 50 vol % of another material. A reparation coating or reparation layer may be incorporated in the mixed membrane structure.

In some embodiments, the matrices may comprise a structure to direct gas flow across a two-dimensional membrane. In some embodiments, the matrices may comprise a three-dimensional structure. The three-dimensional structure may comprise a plurality of microchambers within the larger structure. Each of the plurality of microchambers may be separated from an exit chamber or another of the plurality of microchambers by a membrane (e.g. a ZIF mixed membrane). The gas stream (e.g. methane-containing gas stream) may be directed through one or more microchambers of the plurality of microchambers. The gas stream may pass through the membrane lining the exit to each microchamber into another microchamber or an exit chamber. The membrane may restrict, prevent or otherwise separate the target molecule to be bound (e.g. methane) from exiting one or more of the microchambers. The design of the three-dimensional structure may facilitate the capture of methane from a gas stream in the plurality of microchambers simultaneously. For example, the inlet of the gas stream may be directed to the center of a collection of microchambers. The membranes separating the microchambers may be configured to allow the gas stream to pass through multiple microchambers in series before being direct to an exit chamber or stream. As an example, the gas stream may pass through the series of microchambers comprising the matrix in a linear or radial direction. The membranes delineating the microchambers may comprise the same mixed membrane structure (e.g. ZIF membrane) or one or more of the mixed membrane structures. As an example, the membranes separating the microchambers closest to the gas inlet may have a larger pore size, lower affinity for methane, and/or a higher porosity value to allow for greater gas flux. In the same example, the membranes separating the microchambers closest to the exit chamber or stream may be configured to have a smaller pore size, higher affinity for methane, and/or a lower porosity value to capture restrict, capture or otherwise separate a higher quantity of the target molecule (e.g., methane) from the gas stream.

The matrices may have a higher binding affinity for methane than other components in the gas stream. The membrane may be a single layer or multi-layer membrane. Individual layers of the multi-layer membrane may be made from the same or different materials, for example, a polymeric material. The matrices may comprise a porous structure having a plurality of pores. The pores may be micropores, nanopores, or any combination thereof. The pores may have an average pore size that is greater than or equal to about 1 Å, 3 Å, 5 Å 7 Å, 10 Å, 15 Å, 20 Å, 25 Å, 30 Å, 40 Å, or 50 Å. The pores may have an average pore size that is less than or equal to about 150 Å, 120 Å, 100 Å, 90 Å, 80 Å, 70 Å, 60 Å, 50 Å, 45 Å, 40 Å, 35 Å, 30 Å, 25 Å, or 20 Å. In some cases, the average pore size may be between any of the two values disclosed above, e.g., between 5 Å, and 20 Å. The porous structure may have a selectivity measures as a ratio of methane to one of one or more other gases within the gas stream (e.g. carbon oxides, nitrogen oxides, etc.) For example, the ZIF mixed membrane may comprise hollow ZIF-8 polyhedral nanocrystals distributed in a matrix. In this example, the ZIF structure may have a CO₂/CH₄ selectivity of about 14.

The system may comprise one or more gas monitoring units. One of the gas monitoring units may be configured to measure a composition of the methane-containing gas stream. Another of the gas monitoring units may be configured to measure a composition of the output stream.

The system may comprise a switching unit that can be configured to alternate gas flows into the processing unit or any additional processing units. The switching unit may alternate gas flows between the methane-containing gas stream and an additional gas stream. The switching unit can hereby feed different gas flows, the methane-containing gas stream and the additional gas stream, into the processing unit or any one of the additional processing units.

In some aspects, disclosed herein is a method for processing a methane-containing gas stream by directing the methane-comprising gas stream into a processing unit comprising a matrix and a catalyst, using the matrix to bind at least a portion of methane from the methane-containing gas stream to yield bound methane, and using the catalyst to subject at least a portion of the bound methane to a reaction that decreases an amount or concentration of the bound methane. The reaction in this method may be a combustion reaction of methane. The method may comprise oxidizing at least a portion of the bound methane in the reaction and thereby regenerating the processing unit. The method may yield greater than or equal to about 5%, 10%, 15%, 20%, 25%, 30%, 35%, 40%, 45%, 50%, 55%, 60%, 65%, 70%, 75%, 80%, 85%, 90%, 95% (vol %, or mol %), or more of the methane in the methane-containing gas stream to be bound by the matrix. The method may yield greater than or equal to about 5%, 10%, 15%, 20%, 25%, 30%, 35%, 40%, 45%, 50%, 55%, 60%, 65%, 70%, 75%, 80%, 85%, 90%, 95% (vol %, or mol %), or more of the methane bound to the matrix to be oxidized, or otherwise consumed, by the catalytic reaction.

The method may comprise directing the methane containing gas stream into one or more additional processing units. These one or more additional processing units may be connected to the original processing unit in parallel. The one or more additional processing units may each comprise an additional matrix, an additional catalyst, and/or an additional heating element. The method may comprise using the one or more additional matrices to bind additional methane from the methane-containing gas stream to yield additional bound methane. The method may yield greater than or equal to about 5%, 10%, 15%, 20%, 25%, 30%, 35%, 40%, 45%, or 50% of additional methane in the methane-containing gas stream to be bound to the additional matrix. The method may comprise subjecting the additional bound methane to an additional reaction with aid of the additional catalyst to oxidize at least a portion of the additional bound methane. The method may yield greater than or equal to about 5%, 10%, 15%, 20%, 25%, 30%, 35%, 40%, 45%, 50%, 55%, 60%, 65%, 70%, 75%, 80%, 85%, 90%, 95% (vol %, or mol %), or greater of the bound methane to be oxidized in the additional reaction. The method may comprise repeating the steps of: using the matrix to bind at least a portion of methane from the methane-containing gas stream, using a catalyst to subject at least a portion of the bound methane to a reaction that decreases an amount or concentration of the bound methane, using an additional matrix to bind additional methane from the-methane containing gas stream to yield additional bound methane, and subjecting the additional bound methane to an additional reaction with aid of an additional catalyst to oxidize at least a portion of the additional bound methane.

The method may comprise directing an additional gas stream to the processing unit to react with the portion of bound methane. The method may comprise alternating gas flow to the processing unit and the additional processing unit. The method may alternate between the methane-containing gas stream and the additional gas stream flows to any of the processing units. The additional gas stream may be air, an accelerant (e.g. methane, ethane, butane or propane), or any combination or variant thereof.

In some aspects, disclosed herein is a computer implemented method for promoting a decrease in methane emissions. The method may comprise receiving, by one or more computer processors, methane content data from a quantified destruction system (QDS). The one or more computer processors may be operably coupled to and in communication with the QDS. The QDS may be operably coupled to a methane processing system. The QDS may be part of a methane processing system. The methane content data may comprise a lowered methane content data that describes a quantity of methane that may be oxidized, combusted, or otherwise removed from a gas stream of the methane processing system. The lowered content data may comprise other indicators of the methane processing system (e.g. time of operation, input flow, output flow, accelerant added, air added, operating conditions of the system, catalyst present etc.). The QDS may comprise one or more quantified destruction devices (QDD) (e.g., at least 1, 2, 3, 4, 5, 6, 7, 8, 9, 10, 15, 20, 25, 30, 35, 40, 45, 50, 60, 70, 80, 90, 100, 120, 140, 160, 180, 200, 300, 400, 500, or more). The one or more QDDs may be in operative communication with one another. The one or more QDDs may be in operative communication with one another over a local network (e.g., a local area network (LAN)) or a remote network (e.g., the internet). The one or more QDDs may each be configured to couple with a given methane processing unit comprised in the methane processing system. An individual QDD may be configured to collect gas content data (e.g., methane content data) from a methane processing unit. The methane content data may comprise an amount, quantity or concentration of methane, or a change thereof, in a gas stream (e.g., an input gas stream, an intermediate gas stream, an output gas stream). Based on the gas content data, the QDD may be configured to alter one or more parameters for operation of the methane processing unit. The one or more parameters may comprise a temperature, pressure, gas composition, addition of air or an accelerant to the methane processing unit, or any combination thereof. In some cases, an individual QDD may be in operative communication with one or more methane processing units. In cases where a plurality of QDDs are comprised in the QDS, the QDS may generate a single output of gas content data collected by the plurality of QDDs, or a subset thereof. The QDS may be in operable communication with a database that can store data (e.g. methane content data or lowered methane content data) generated by the QDS or one or more QDDs within the QDS. The QDS may lower or have lowered (e.g., via the methane processing system) a methane content of a gas stream to yield a lowered methane content, may measure the methane content data, and may transmit the methane content data to the computer processor(s). The methane content data may comprise the methane content of the gas stream. The method may further comprise encrypting a data block comprising lowered methane content data to generate encrypted data. The data block may comprise lowered methane content data derived from the methane content data and a QDS identifier. The QDS identifier may be unique to the QDS among a plurality of QDS's. The data block may further comprise data on geo-positioning information of the QDS, and a time stamp. In some cases, a QDS is a system that has the characteristics, or can be configured to perform the functions, herein described. As described in this disclosure, a QDD may be any device that has the characteristics, or can be configured to perform the functions, herein described.

The method may further comprise storing the encrypted data on a blockchain database. A blockchain database is generally a digitized, distributed ledger, in which entries or transactions are recorded. The entries or transactions are marked and secured by encrypted signatures such that the historical record of entries or transactions is impervious to tampering. The entries or transactions are cross checked against a plurality of copies of the ledger on a plurality of servers, thus no single server can alter past entries or transactions. The blockchain database may comprise a network of distributed nodes in operative communication with one or more computer processors. Blockchain may be applied to allow execution of smart contracts relating to digital token transactions, (e.g. buying, selling, or exchanging carbon credits). Smart contracts may comprise self-executing machine-readable instructions that can store state information and can be stored on the blockchain database. The smart contract may have the ability to read its internal storage, store data by writing to its internal storage, or send and receive messages with other smart contracts to trigger an execution of code in other applications. The smart contract may include machine-readable instructions to access the internal storage of the smart contract and write data to the internal storage reflecting a transaction of digital tokens. For example, in a smart contract that governs the sale of a digital token associated with a carbon credit, the smart contact may include machine-readable instructions to access its internal storage, read the storage of a message received by the smart contract, and process the data to reflect a transaction. In some embodiments, the message received by the smart contract contains data that reflects an offer, counter-offer, acceptance, or rejection of an offer in relation to a transaction of digital tokens. The smart contract writes this new data of the completed or incomplete transaction to the internal storage of the smart contract and the updated smart contract may be stored as an event or new block in the blockchain. In communicating with other applications, the smart contract can execute code in those other applications that reflect the terms of the transaction. For example, in a purchase of digital tokens, (e.g. carbon credits), the executed smart contract for the purchase of the digital tokens can execute the appropriate code in an application that transfers other forms of currency or other digital tokens or assets from the purchaser to the seller. In some cases, a new block on the blockchain is created by updating a smart contract that details a transaction of digital tokens. Each new block may be cryptographically validated and linked to earlier blocks and serve as an immutable record of the previous smart contract executions.

The QDS may comprise a quantified destruction device (QDD). The QDS may comprise a unit that reacts, consumes, or eliminates, captures, or otherwise removes a substance (e.g. methane, carbon oxides (COx), nitrogen oxides (NOx), or any other volatile organic compounds (VOCs)) from a stream, gas flow, processing unit, matrix, or other location. The QDS or QDD may comprise a catalytic reactor, biological filtration unit, thermal oxidizer, or one or more additional units.

The gas stream of this method may be an input gas stream received by the QDS. The methane content data may comprise the methane content of the input gas stream and a methane content of an output gas stream generated by the QDS. The QDS may further lower an amount or concentration of one or more additional components in the input gas stream. The QDS may further track the amount or concentration of the one or more additional components. The one or more additional components may comprise carbon oxides (COx), nitrogen oxides (NOx), any other volatile organic compounds (VOCs), or any combination thereof. The QDS may measure gas content data comprising the amount or concentration of the one or more additional components and transmit the gas content data to one or more processors. For example, the QDS may lower the concentration of methane in a methane-containing gas stream and transmit the gas content data of the resulting methane concentration, along with the carbon dioxide gas content data, to a blockchain-based ledger of one or more computer processors. The lowered methane content data may comprise data on emission reductions of the methane content between the input gas stream and the output gas stream. The method may further comprise tracking and recording the emission reductions from the QDS. The method may comprise tracking an exchange of emission units based on the lowered methane content data between users of the blockchain database. The method may comprise processing, by one or more computer processors, the lowered methane content data to determine a carbon credit. The method may comprise issuing the carbon credit(s) as digital tokens of the blockchain database to a user associated with the QDS. The encrypted data stored on the blockchain database may comprise data on the issuing of the digital tokens. The method may comprise tracking a transaction of the carbon credit(s), or digital tokens, between users of the blockchain database. The transaction may be digitally signed by a trusted third-party. The method may comprise disseminating a digital wallet to the QDS or a user account associated with the QDS. The method may comprise storing the digital wallet in the blockchain database. The method may comprise recording and validating new entries on the blockchain database.

In some aspects, disclosed herein is a system for promoting a decrease in methane emissions. The system may comprise a quantitative destruction system (QDS) and one or more computer processors in operative communication with the QDS. The QDS may be associated with a QDS identifier unique to the QDS. The QDS may be configured to lower a methane content of a gas stream to yield a lowered methane content. The QDS may be configured to measure methane content data comprising the methane content of the gas stream. The QDS may be configured to transmit the methane content data. One or more computer processors may be individually or collectively configured to receive the methane content data, encrypt a data block to generate encrypted data, and store the encrypted data on a blockchain database. The data block may comprise the QDS identifier and lowered methane content data derived from the methane content data. The QDS may be one of a plurality of QDSs in fluid communication with a plurality of input gas streams. One or more computer processors may be in operative communication with the plurality of QDSs. Each QDS may be associated with a respective QDS identifier unique to each QDS amongst the plurality of QDSs. The QDS may comprise one or more of a methane measurement module, a carbon dioxide (CO₂) output measurement module, a geo-positioning module, and a time recording module. The blockchain database may comprise a platform for recording and validating new entries on the blockchain database.

One or more computer processors may be further configured to process the lowered methane content data to determine a carbon credit. One or more computer processors may be further configured to issue the carbon credit(s). One or more computer processors may be further configured to process the lowered methane content data to determine a carbon credit. One or more computer processors may be further configured to issue the carbon credit(s) as digital tokens of the blockchain database to a user associated with the QDS. One or more computer processors may be further configured to retire or eliminate a digital token according to predetermined criteria. The predetermined criteria may comprise a threshold for the elapsed time from the data the methane content is reduced by the QDS. The predetermined criteria may comprise a voluntary decision by an owner of a digital token. The encrypted data stored on the blockchain database may comprise data on issuance of the digital tokens. The encrypted data on the blockchain database can be accessible by a trusted third-party. The blockchain database may comprise an additional platform. The additional platform can track a transaction of the carbon credit(s) between users of the blockchain database. The transaction may be digitally signed by a trusted third-party. The blockchain database may comprise a platform for tracking an exchange of emission units, based on the lowered methane content data, between users of the blockchain database. The system may comprise a blockchain verification module configured to generate one or more binary strings to identify a transaction on the blockchain database. A first binary string of one or more binary strings can identify a current blockchain node as a transaction recipient. A second binary string of one or more binary strings can identify the current blockchain node as a transaction sender.

### Computer Systems

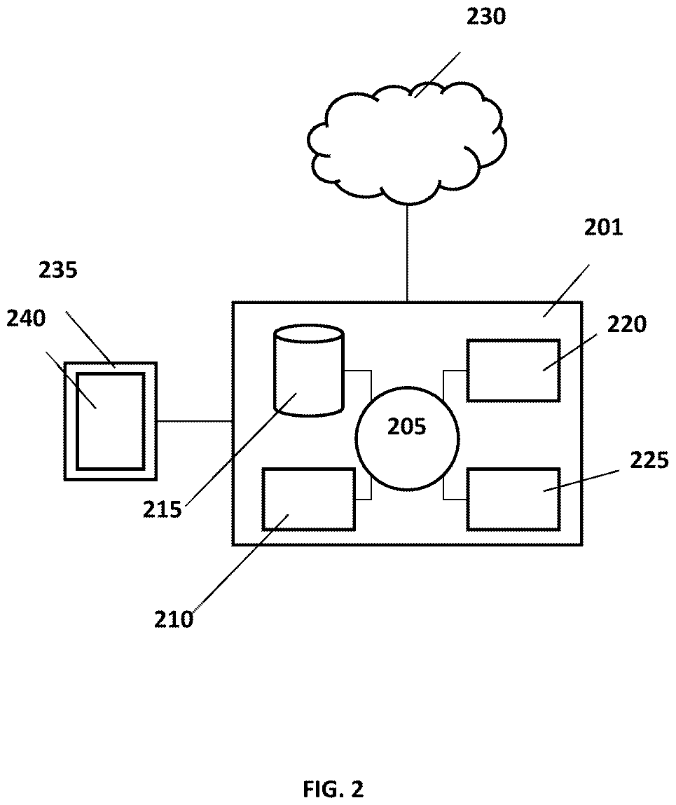

The present disclosure provides computer systems that are programmed to implement methods of the disclosure. FIG. 2 shows a computer system 201 that is programmed or otherwise configured to promote the reduction of methane emissions. The computer system 201 can regulate various aspects of methods and systems of the present disclosure, such as, for example, various processing units of the system including the biological filtration unit, the thermal oxidizer, the condenser, the one or more additional units or any combination thereof. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, access to the history of the carbon credit or digital token transactions on the blockchain database or access to generate a transaction of the carbon credits or digital tokens on the blockchain database. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205.

Example—System and Method for Biological Remediation of Methane-Containing Streams

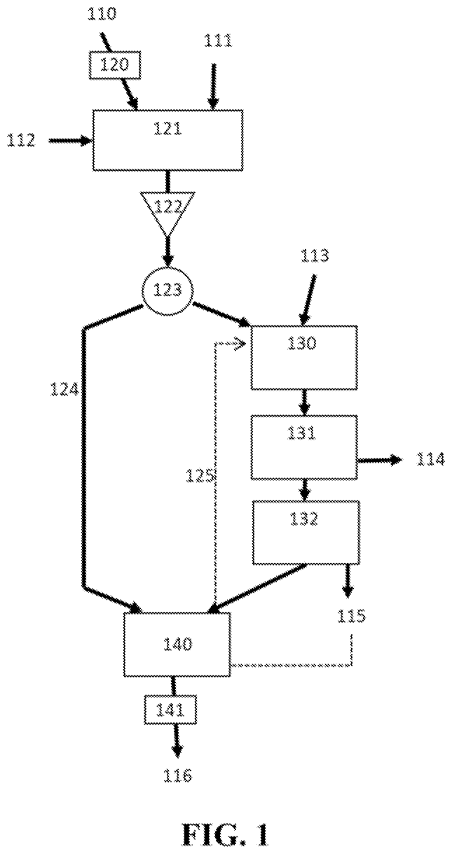

As shown in FIG. 1, an example system of the present disclosure comprises a gas inlet 110, an air inlet 111, and an accelerant inlet 112. A methane-containing gas stream is directed into the system via the gas inlet. The methane-containing stream comprises at least 10 vol % methane. The methane-containing stream also comprises carbon oxides (e.g., carbon monoxide, carbon dioxide etc.), nitrogen oxides, and other volatile organic compounds (VOCs). A gas monitoring unit 120 in the system is used to measure a composition of the methane-containing gas stream entering into the system via the gas inlet. The composition includes a concentration of methane, carbon oxides, nitrogen oxides, and/or VOCs in the gas stream. The methane-containing gas inlet, air inlet and accelerant inlet are in fluid communication with a gas mixing unit 121. The gas mixing unit mixes the methane-containing input stream, air and an accelerant into a mixed gas stream. The mixed gas stream comprises at least 10 vol % methane. The mixed gas stream is directed to a blower 122. Downstream of the blower, the system further comprises a gas switch 123. Depending upon, for example, the composition of methane-containing input gas measured by the gas monitoring unit, the methane-containing input gas stream is sometimes directed to the gas switch without passing through the gas mixing unit, and the methane-containing gas stream is not mixed with air or an accelerant. The gas switch directs the gas stream to a gas bypass 124, or to a humidifier 130. The gas switch and bypass are configured to regulate gas flow within the system. When the switch is set in a first position, the methane-containing input gas stream, or a portion thereof, is directed to a thermal oxidizer directly without passing through the humidifier, biological filtration unit, or condenser. When the switch is set in a second position, the methane-containing input gas stream, or a portion thereof, is directed to a humidifier. The humidifier is configured to receive a water stream and permit the gas stream to be in contact with the water stream to generate a humidified gas stream. The humidified gas stream contains at least 10 vol % methane, and at least 5 vol % water. The humidified gas stream, or a portion thereof, is directed to a biological filtration unit 131, downstream of the humidifier. The biological filtration unit processes the humidified gas stream (or a portion thereof) to produce a processed gas stream and a biomass materials outlet 114. The processed gas stream has 5 vol % methane. The biomass materials outlet has a heating potential of at least 1 Btu/kg. The biomass materials are optionally directed to one or more subsequent units for further processing. The processed gas stream is directed from the biological filtration unit to a condenser 132. The condenser is configured to yield, from the processed gas stream or a portion thereof, a water stream 115 and a remaining gas stream. The remaining gas stream contains at least 0.5 vol % methane. The remaining gas stream is directed to a thermal oxidizer 140. As discussed above, in some modes of operation, the input gas stream is routed directly to the thermal oxidizer without passing through the biological filtration unit, condenser, or humidifier that are upstream of the thermal oxidizer. Water and heat generated in the thermal oxidizer are recycled 124, e.g., to the humidifier. The thermal oxidizer generates an output stream and expels at least a portion of the output stream from the system as an exhaust stream 116. The output stream contains 0.1 vol % methane. An additional gas monitoring unit 41 is used to monitor compositions of the output stream and the exhaust stream. The compositions include concentrations of methane, carbon oxides, nitrogen oxides, and/or VOCs in the output stream and exhaust stream. The system is operably coupled to and controlled by a computer control system.

Example—System and Method for Catalytic Remediation of Engine Exhaust

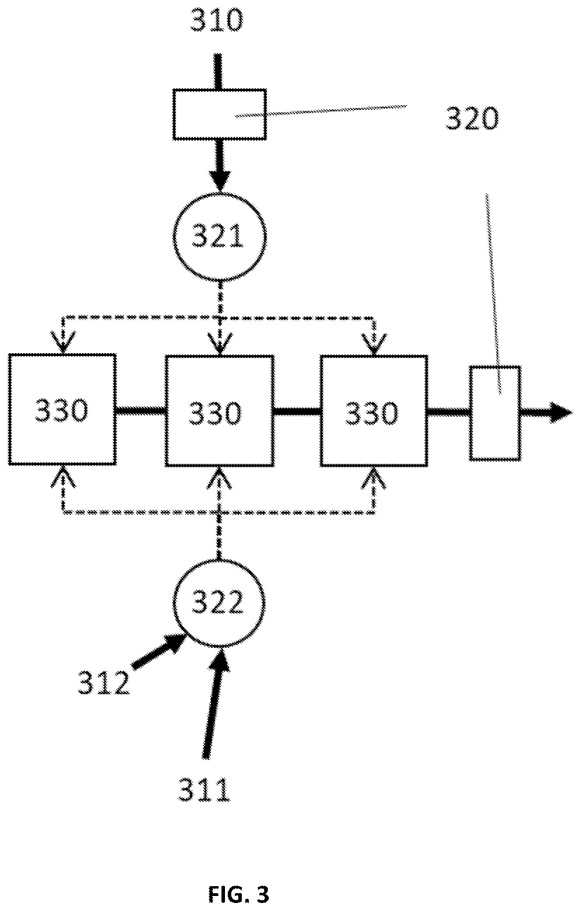

As shown in FIG. 3, an example system of the present disclosure comprises an inlet configured to receive a stream. The stream is an exhaust stream from an engine 310. The stream comprises methane, along with carbon oxides (e.g., carbon monoxide, carbon dioxide), nitrogen oxides, and other volatile organic compounds (VOCs). A composition of the engine exhaust stream is measured by a gas monitoring unit 320. The gas monitoring unit measures a composition of the gas stream. The composition includes a concentration of methane, carbon oxides, nitrogen oxides and other VOCs within the gas stream. The gas stream is directed to a first gas switch 321. The gas switch directs the gas flow to one or more (e.g. three) processing units 330. The system further comprises an air inlet 311 configured to receive air and an accelerant 312. The accelerant comprises methane, ethane, propane, butane, methanol, ethanol, propanol, butanol, formate, formaldehyde, acetate, acetaldehyde or any combination or variant thereof. The air and the accelerant are directed to a second gas switch 322. The second gas switch directs the air and/or accelerant gas stream to one or more (e.g. three) processing units 330. The one or more processing units each comprise one or more of a catalyst, a heating element, and a matrix for binding methane. Within each of the processing units, the methane in the gas stream is bound within the matrix. The remaining gases in the gas stream flow are directed out of the processing unit. With the aid of the heating element and catalyst, the methane bound within the matrix of each processing unit is subjected to a reaction that combusts, oxidizes, or otherwise removes the methane from each matrix within each processing unit. This process leaves each processing unit thereby regenerated and available to capture sequential methane from sequential flow of the gas stream, as directed by the first or second gas switching unit. Before exiting the system, the one or more processing units direct their output streams in series to a gas monitoring unit 320. The gas monitoring unit measures the composition of the gas stream.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-119. (canceled)

120. A computer-implemented method for promoting a decrease in methane emissions, comprising:
(a) receiving, by one or more computer processors in operative communication with a quantified destruction system (QDS), methane content data from said QDS, wherein said QDS:
(i) lowers a methane content of a gas stream to yield a lowered methane content;
(ii) measures said methane content data comprising said methane content of said gas stream; and
(iii)transmits said methane content data to said one or more computer processors; and
(b) encrypting, by said one or more computer processors, a data block comprising lowered methane content data derived from said methane content data and a QDS identifier, to generate encrypted data.

121. The method of claim 120, further comprising storing said encrypted data in a database.

122. The method of claim 120, wherein said QDS comprises a biological filtration unit.

123. The method of claim 122, wherein said biological filtration unit comprises a bacterial consortium.

124. The method of claim 123, wherein said bacterial consortium comprises a methanotroph.

125. The method of claim 120, wherein said QDS comprises one or more quantified destruction devices (QDD), wherein said one or more QDDs is configured to alter a parameter.

126. The method of claim 125, wherein said parameter is selected from the group consisting of a temperature, a pressure, a gas composition, a quantity of an addition of air or an accelerant to the methane processing unit, and any combination thereof

127. The method of claim 120, wherein the QDS further lowers an amount of an additional component in said input gas stream.

128. The method of claim 127, wherein said additional component is selected from the group consisting of carbon dioxide, nitrogen oxides, other volatile organic compounds, and any combination thereof.

129. The method of claim 120, wherein said gas stream is an input gas stream received by said QDS.

130. The method of claim 129, wherein said methane content data comprises said methane content of said input gas stream and a methane content of an output gas stream generated by said QDS.

131. The method of claim 120, wherein said QDS comprises a catalytic reactor.

132. A system for promoting a decrease in methane emissions, comprising:
(a) a quantitative destruction system (QDS) configured to
(i) lower a methane content of a gas stream to yield a lowered methane content;
(ii) measure methane content data comprising said methane content of said gas stream; and
(iii) transmit said methane content data, wherein said QDS is associated with a QDS identifier unique to said QDS; and
(b) one or more computer processors in operative communication with said QDS, wherein said one or more computer processors are individually or collectively configured to:
(i) receive said methane content data; and
(ii) encrypt a data block comprising said QDS identifier and lowered methane content data derived from said methane content data, to generate encrypted data.

133. The system of claim 132, wherein said QDS comprises a biological filtration unit.

134. The system of claim 133, wherein said biological filtration unit comprises a bacterial consortium.

135. The system of claim 134, wherein said bacterial consortium comprises a methanotroph.

136. The system of claim 132, wherein said QDS comprises one or more quantified destruction devices (QDD), wherein said one or more QDD is configured to alter a parameter.

137. The system of claim 136, wherein said parameter is selected from the group consisting of a temperature, a pressure, a gas composition, a quantity of an addition of air or an accelerant to the methane processing unit, and any combination thereof

138. The system of claim 132, wherein said gas stream is an input gas stream received by said QDS.

139. The system of claim 132, wherein said one or more computer processors are further configured to process said lowered methane content data to determine a carbon credit.
